(19) United States
(12) Patent Application Publication
(10) Pub. No.: US 2005/0085908 A1
(43) Pub. Date: Apr. 21, 2005

(54) New ophthalmic lens materials with high refractive index and biocompatible surface

(76) Inventors: Yu-An Chang, Irvine, CA (US); Jim-Son Chou, Irvine, CA (US)

Correspondence Address: Yu-An Chang, Ph.D., 3631 Hamilton Street, Irvine, CA, 92614, US

(21) Appl. No.: 10/688,757

(22) Filed: Oct. 20, 2003

(51) Int. Cl. A61F 2/16

(52) U.S. Cl. 623/6.56; 526/292.5; 526/312; 526/307.5; 526/318; 526/323.1; 526/318.1

(57) ABSTRACT

New aryl acrylic core polymers processed with a thin layer of biocompatible hydrophilic polymers on the surface for the medical devices used in eye surgeries and other medical applications and medical devices are disclosed.

### TECHNICAL FIELD OF THE INVENTION

The present invention generally relates to a serial of new ophthalmic lens materials which have high refractive index and biocompatible surface that can be used for foldable Intraocular Lenses (IOLs).

These materials of the present invention are Improved soft, foldable acrylic lens materials but which are also useful as other ophthalmic devices, such as contact lenses, keratoprostheses, and corneal rings or inlay as well as other medical devices. These materials contain two principal components: (1) The major core polymers are aryl acrylic hydrophobic polymers and (2) a thin layer of a biocompatible hydrophilic polymer processed onto the core polymer. This surface processed polymer is less than 1% by weight. The core polymer materials of the present invention are copolymers comprising at least about 90% of hydrophobic aryl acrylic monomers. The remainder of the material comprises up to 10% by weight of one or more additional components, such as cross-linking monomers, as well as other materials, for example: UV-light absorbing, and other light absorbing components if there is a need of them.

### BACKGROUND OF THE INVENTION

Due to the population aging, more patients need intraocular lens (IOLs) surgeries. Especially for senior patients, refractive lens exchange and implantation of multifocal IOLs can provide the patients with excellent functional vision after cataract surgeries. Phakic IOL implantation is another procedure of growing interest in the USA. It has been studied in South America and Europe with successes. IOLs have been used for both myopic and hyperopic eyes. Technical refinement and improvement of the design of these lenses will make this surgery an alternative to current refractive methods, i.e., wearing glass or contact lenses.

Early IOLs made from polymethylmethacrylate (PMMA) were rigid. They required a large incision (greater than 6 mm) in order to be inserted in the eye. Thus cause a prolonged and uncomfortable healing process. In order to reduce healing time and potential complications. Search of soft IOLs that could be folded and inserted through a considerably smaller opening (on the order of 4.0 mm or less) has been carried on for many years. However, folding an IOL for small incision implantation is difficult to accomplish due to the intensely conflicting physical demands required of the polymers used to make such medical implants, for example, optical clarity, non-sticky surfaces, stability and biocompatibility, sufficient flexibility for folding with sufficient stability to resist damage and distortion during folding.

Recent advances in small-incision cataract surgeries and other foldable IOLs require soft, foldable polymer materials suitable for use in artificial lenses. There are three major categories of these polymers, i.e., hydrogels, silicones, and acrylics.

Most hydrogel polymers have a relatively low refractive index, making them less desirable than other materials because of the thicker lens optic necessary to achieve a given refractive power. Silicone polymers possessed excellent optical clarity and had excellent elasticity. They can have a higher refractive index than hydrogels and were generally biocompatible, but tend to unfold explosively and damage critical nerve cells in the eyes, the corneal endothelium and/or rupture the natural lens capsule during insertion and unfolding. Consequently, they required special implantation tools and techniques. They also have problems of latent biocompatibility concerns. Adherence of silicon oil used in vitreoretinal surgery to an IOL that had been previously implanted in the eye undergoing treatment is a serious problem for both patients and eye surgeons. The irreversible silicone oil adhesion to the silicone IOL surface not only diminished the patient's visual acuity, but also hindered the surgeon's ability to view inside patients' eyes. The biocompatibility of an IOL material can be evaluated by the opacification pattern on the anterior capsule covering the IOL optic. Many literature reports indicated that hydrophobic, less biocompatible IOL make of silicone polymer material induces more intense anterior capsule opacification (ACO) than poly (methyl methacrylate) (PMMA) or acrylic materials, although histological and ultrastructural examinations have not been systematically carried out. Therefore, new and better IOL materials are still urgently sought-after.

Recently, Bausch & Lomb markets a hydrophilic polymethacrylate hydrogel IOL (HYDROVIEW^{R}), which is the most biocompatible among all fordable IOL's. However, calcium deposit, and Lens epithelial cells (LECs) proliferation on the anterior capsule are worse than other silicone and acrylic IOLs while extracellular matrix accumulation appeared immature and less compare to other IOLs. Many other acrylic polymers have been developed and evaluated for foldable IOL fabrication. The majority of them are copolymer mixes of multiple monomers in order to produce the desired combination of properties possessed by each monomer component. The ideal ocular implant or IOLs must be optically clear and permanently remain so after implantation. The refractive index preferred to be greater than the first generation silicon IOL's (i.e., 1.43) and the lens must be stably elastic and capable of stretching to 150% of its pre-stretch size before breaking (elongation factor). The implant must be soft enough to allow easy pre-insertion folding and it must have a non-sticky surface so that the inserted lens will unfold in a predictable manner without requiring further or difficult manipulation.

These demands are extremely difficult to be satisfied in a single polymer material. For example, polymers with less sticky surfaces are often too hard and can crack when folded. While softer polymers that fold easily, usually have sticky surface, making them difficult to handle and complicating the implantation and post insertion unfolding. Besides, the ideal ocular implant must have a stable elastic structure that will not be damaged, distorted, or destroyed by folding, while at the same time retaining all of the optical qualities required to function as a successful implant, lens, or corneal replacement.

Acrylic polymers have a high refractive index and unfold more smoothly or controllably than silicone materials. U.S. Pat. No. 5,693,095 discloses high refractive index, acrylic materials suitable for use as an IOL material. The IOLs made of these acrylic materials can be rolled or folded for insertion through small incisions. U.S. Pat. No. 5,331,073 and U.S. Pat. No. 5,290,892 also discloses soft acrylic IOL materials. These materials contain acrylic monomers. One of the monomers has a refractive index of at least about 1.50. The other monomer has a glass transition temperature less than about 22° C. These IOL materials also contain cross-linking monomers and/or contain a hydrophilic monomer. These hydrophilic and cross-linking monomers preferably have a total of less than about 15% by weight. U.S. Pat. No. 6,271,281 discloses an intraocular lens formed from an optically clear, high refractive index, low-tack, crosslinked homopolymer.

Many medical devices need to have surface modification for eliminating or reducing blood activation, or other medical effects. For example, Tu, et al. disclosed in the US patent (U.S. Pat. No. 6,506,398) about a vascular graft comprising Vascular Endothelial Growth Factor (VEGF) and/or Platelet Derived Growth Factor (PDGF) for enhanced site-specific angiogenesis and methods thereof are disclosed. At least one VEGF, PDGF or angiogenesis factor is incorporated into the vascular graft to facilitate enhanced angiogenesis so as the cells are stimulated to migrate to environments having higher concentration of growth factors and start mitosis.

The current invention with the hydrophilic surface processing provides a unique opportunity for covalent binding of chemicals, drugs, and antigens onto the surface of the medical devices. It can be used for specific cell attachment such as epithelium cells to prevent negative immune respond to the foreign surface of the medical devices. The hydroxyl functional groups of the biocompatible hydrophilic polymer on the surface of the core polymer can be activated using various conjugation chemistries, such as tosyl chloride and 4-fluorobenzenesulfonyl chloride, etc. thus can covalently attached with various commercial available drugs for medical applications. This modified surface also can be used for cell attachment for the physiological and biological benefits.

### SUMMARY OF THE INVENTION

New improved soft, foldable polyacrylic lens materials disclosed in this invention are particularly suitable for use as IOLs, and also useful as other ophthalmic devices, such as contact lenses, keratoprostheses, as well as corneal rings or inlays. These materials contain two principal components: (1) The major core polymers are hydrophobic aryl acrylic polymers and (2) a thin layer of a biocompatible hydrophilic polymer processed on the core polymer. This surface polymer is less than 1% by weight. They are uniquely processed onto the surface of the core polymer under very mild conditions, i.e., the process only requires two simple steps: (1) soaking the core polymer in the biocompatible hydrophilic polymer solution at room temperature overnight, then (2) the soaked core polymer materials can be incubated at temperature between 40 to 70° C. in an aqueous salt solution for 3 to 18 hours depend upon the incubation temperature. The major core polymer materials of the present invention are copolymers comprising at least about 90% of hydrophobic aryl acrylic monomers. The remainder of the material comprises up to 10% by weight of one or more additional components, such as cross-linking, UV-light absorbing, and blue-light absorbing components. Among other factors, the present invention is also based on the finding that, unlike other acrylic copolymers useful as IOL materials, the core hydrophobic aryl acrylic polymers of the present invention after coating with a thin layer of a biocompatible hydrophilic polymer can be substantially free of tissue growth and protein deposit in a physiologic environment of eyes. As a result, cell migration between the back of the IOL and the posterior capsule of the eye [a process that often results in posterior capsule opacification (PCO) which may lead to blindness] can be significantly reduced On the contrary, patients with IOLs made from conventional polymers may be more susceptible to cell migration and result in PCO.

Further, as known in the art, a common, non-invasive surgical procedure for eliminating posterior capsule opacity is to use a laser, such as an Yittrium Aluminum Garnet or YAG laser, to restore the patient's vision. This procedure, known as YAG Capsulotomy, sometimes causes lens damage on a conventional acrylic IOL when they were inadvertently struck by the YAG laser during the capsulotomy. This can cause damage ranging from pitting of the lens to complete fracturing of the lens require its surgical removal and replacement. Quite the opposite, IOLs made from the present invention, in addition to being less susceptible to PCO, can be less susceptible to laser damage as well. In the rare event that PCO does occur in association with the lenses of the present invention, it is believed that the consistency of the core hydrophobic aryl acrylic polymers of the present invention will render IOLs made from the present invention significantly less susceptible to the damaging effects of YAG lasers. Thus, pitting and cracking from misdirected lasers will be significantly reduced or avoided. Therefore, it is believed that stably soft, elastic IOLs manufactured from the new polymers of the present invention will significantly reduce the occurrence of PCO as well as reduce the occurrence of lens damage from laser capsulotomnies, if later required. This, in turn, will significantly minimize the unnecessary suffering of patients and surgery complications. The new materials of this invention can significantly eliminate unnecessary medical services, thus considerably reduce medical expenses.

Adherence of silicone oil used in vitreoretinal surgery to an IOL that had been previously implanted in the eye can cause a major problem for both patients and eye surgeons. The degree of adherence was primarily depended upon the hydrophilicity of the IOL surface. The more the hydrophilic, the less the silicone oil adherence. For example, most severe adherence occurs with the silicone IOL, other IOLs made from acrylic polymers also have this problem although at a less degree, while very hydrophilic hydrogel IOL and heparin surface modified (HSM) IOL are probably exempted from this trouble. Therefore, the IOL and other eye medical devices manufactured using the present invention, which processed with a thin layer of hydrophilic polymer on their surface can be immune from this problem.

### BRIEF DESCRIPTION OF THE DRAWINGS

Additional objects and features of the present invention will become more apparent and the invention itself will be best understood from the following Detailed Description of Exemplary Embodiments, when read with reference to the accompanying Figures, and Tables.

FIG. 1 is a schematic diagram of the general chemical structure of the hydrophobic aryl acrylic monomers, which can be used for the preparation of the core polymers.

FIG. 2 is a schematic diagram of the general chemical structure of the monomer for the preparation of the biocompatible hydrophilic surface coating polymers.

### DESCRIPTION OF THE INVENTION

The present invention contemplates the new ophthalmic lens materials, which have high refractive index and biocompatible surface that can be used for foldable Intraocular Lenses (IOLs). They are also useful as other ophthalmic devices, such as contact lenses, keratoprostheses, and corneal rings or inlay, as well as other eye implant surgeries and medical devices under development.

The present invention utilizes hydrophobic aryl acrylic monomers and minor amount of appropriate cross-linking monomers to produce optically clear, high refractive index, low-tack core polymers. These core polymers can be processed to the forms and shapes required for various medical devices for eye surgeries, such as ocular implants, including intraocular lenses (IOLs), corneal implants or overlays, etc. The medical devices prepared from these core polymers can then be processed with a thin layer of hydrophilic surface coating polymers. In contrast to the prior art, IOLs made from these optically clear, high refractive index, low tack biocompatible surface processed polymers of the present invention are stable, elastic and can be rolled or folded without destroying, distorting, or damaging the shape or resultant function of the lenses.

Following the teaching of the present invention, these polymers have unique physical and chemical properties including glass transition temperatures of equal to or less than about 15° C., refractive index of greater than 1.50, and elongations at break of at least 150%. These stable, elastic, optically clear, high refractive index, low tack polymers of the present invention are particularly well suitable for use in medical devices mentioned above.

The IOLs made in accordance with the teachings of the present invention can be shape by machines or cast in configurations providing IOLs with sharp edges, which is medically desirable. Moreover, IOLs made in accordance with the teachings of the present invention can be thinner than known foldable lenses and can be rolled or folded for insertion through small incisions in the eye of approximately 3 mm or even less. Once inserted in the eye, the low tack surfaces of the IOLs provided by the present invention permit these IOLs to unfold in a controllable manner that minimize the possibility of eye damage thus eliminate the need of additional post insertion manipulation of the lenses by the implanting surgeon.

In general, the hydrophobic aryl acrylic monomers of the core polymers of the present invention include, but are not limited to the following commercially available aromatic monomers: phenoxyethylacrylate, 2-phenylethylacrylate, 3-phenylethylacrylate, 4-phenylethylacrylate, etc. The stable cross-linkers of the present invention include, but are not limited to the following commercially available cross-linking monomers: divinylbenzene, ethylene glycol dimethacrylate, bis(4-(2-acryloylethoxy) phenyl) methane, bis(2-acryloylalkylphenyl) propane, etc.

In one exemplary embodiment of the present invention the aryl acrylic polymer of the present invention are formulated to contain from about 99 to 90% aryl acrylic monomer and the stable cross-linkers are in a concentration of between approximately 1 to 10%.

1. A method of processing for intraocular implants and medical devices using hydrophobic core copolymers with a surface coating of biocompatible hydrophilic polymer.

2. The method of claim 1, wherein said the Intraocular implants are the foldable Intraocular Lenses (IOL's) and other ophthalmic implants such as implantable contact lenses, keratoprostheses, and corneal rings or inlay.

3. The method of claim 1, wherein said the medical devices are catheters, vascular grafts or stents, artificial joints, medical devices for blood oxygenation, dialysis, coronary artery implants, femorofemoral artery implants, femoral-poplitial artery implants, femoro-tibial artery implants, fibular artery implants, plantar artery implants, dorsalis-pedis artery implants, arterial-venous fistulae, and venous implants.

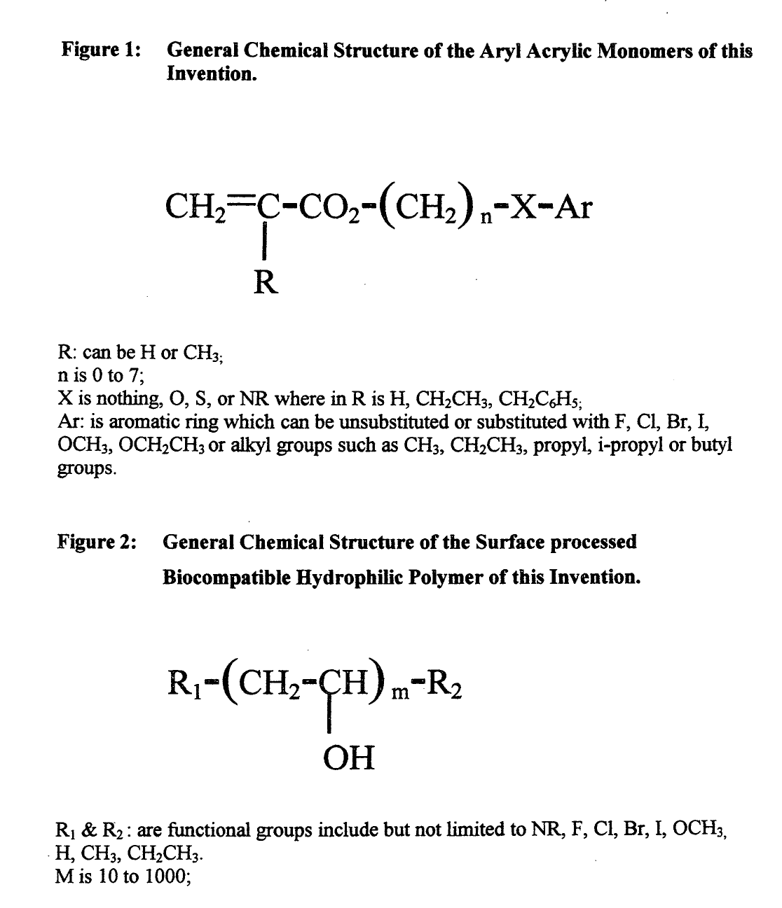

4. The method of claim 1, wherein said the hydrophobic core copolymers having an elongation of at least 150%, comprising a total of at least 90% by weight of two principal monomers, wherein one principal core monomer is an aryl acrylic hydrophobic monomer described in FIG. 1 wherein: R is hydrogen or methyl group, n is 0 to 7. Ar is any aromatic ring which is unsubstituted or substituted with F. Cl, Br, I, OCH₃, OCH₂CH₃, or Alkyl groups such as CH₃, CH₂CH₃, propyl, i-propyl or butyl groups; X is nothing, O, S, or NR where in R is H, CH₂CH₃, CH₂C₆H₅. The other monomer, present in an amount not greater than 10% of the aryl acrylic hydrophobic monomer, is a cross-linking monomer.

5. The method of claim 1, wherein said the copolymers have UV-light absorbing, and/or other light absorbing components added into the core hydrophobic monomers.

6. The method of claim 1, wherein said the hydrophobic core copolymers are processed to the desired forms and shapes and then a biocompatible hydrophilic polymer is processed onto the core acrylic polymer. The general chemical structures of these biocompatible hydrophilic monomers is described in FIG. 2 wherein R₁ & R₂ are functional groups such as NR, F, Cl, Br, I, OCH₃, OCH₂CH₃, or Alkyl groups such as CH₃, CH₂CH₃, propyl, i-propyl or butyl groups; M is 10 to 1000.

7. The method of claim 1, wherein the intraocular implants can be prepared by individually machining or produced by injection molding.

8. The method of claim 1, wherein the surface coating of biocompatible hydrophilic polymer can be activated using conjugation chemical reactions for the covalently attachment of commercially available pharmacologically active chemicals.

9. The method of claim 8, wherein said the pharmacologically active chemicals are anti-coagulant drugs, anti-cancer drugs, Vascular Endothelial Growth Factor (VEGF) and/or Platelet Derived Growth Factor (PDGF) which include, but not limited to heparin, Taxol, and angiogenesis factor is selected from the group consisting of VEGF, VEGF 2, bFGF, VEGF121, VEGF165, VEGF189, VEGF206, PDGF, PDAF, TGF-B, PDEGF, PDWHF.

10. The method of claim 8, wherein said the bio-compatible surface processed copolymers can covalently attached with cells from specific tissue or cell lines to create special biological effects, such as endothelium cells to reduce blood activation, and other unwanted or harmful biological activities.
